(19) United States
(12) Patent Application Publication
(10) Pub. No.: US 2023/0061859 A1
(43) Pub. Date: Mar. 2, 2023

(54) PYRIDO[2,3-D]PYRIMIDINE HIV-1 REVERSE TRANSCRIPTASE INHIBITOR WB3, PREPARATION METHOD AND APPLICATION THEREOF

(71) Applicant: SHAN DONG UNIVERSITY, Jinan (CN)

(72) Inventors: Xinyong LIU, Jinan (CN); Dongwei KANG, Jinan (CN); Peng ZHAN, Jinan (CN); Zhao WANG, Jinan (CN); Guoxiong LI, Jinan (CN)

(21) Appl. No.: 17/757,824

(22) PCT Filed: Mar. 3, 2020
(86) PCT No.: PCT/CN2020/077565

§ 371 Date: Jun. 21, 2022

(30) Foreign Application Priority Data
Feb. 21, 2020 (CN) 202010105708.9

(51) Int. Cl. C07D 471/04

(52) U.S. Cl. CPC C07D 471/04

(57) ABSTRACT

The invention relates to pyrido[2,3-d]pyrimidine compound WB3 and its preparation method and application. The structure of WB3 is as follows, and the invention also relates to a pharmaceutical composition of WB3. Moreover, the invention also provides applications of the WB3 in the preparation of drugs for the treatment and prevention of HIV.

### FIELD OF THE INVENTION

The invention belongs to the technical field of organic compound synthesis and pharmaceutical applications, in particular to pyrido[2,3-d]pyrimidine HIV-1 reverse transcriptase inhibitors WB3 and its preparation method and application thereof.

### BACKGROUND OF THE INVENTION

HIV-1 reverse transcriptase (RT) plays a key role in the replication cycle of HIV-1, making it an important target for anti-HIV-1 drug development. Reverse transcriptase inhibitors acting on RT are mainly divided into nucleoside reverse transcriptase inhibitors (NRTIs) and non-nucleoside reverse transcriptase inhibitors (NNRTIs). In particular, NNRTIs are widely used in the highly active antiretroviral therapy (HAART) because of their potent antiviral activity and high selectivity. However, the drug resistance and poor pharmacokinetic properties of NNRTIs limited their clinical application. Therefore, the development of next-generation NNRTIs with greater potency, improved drug-resistance profiles, and less toxicity is still required

### SUMMARY OF THE INVENTION

In view of the deficiencies of the prior art, the present invention provides a pyrido[2,3-d]pyrimidine derivative WB3 and a preparation method thereof. The invention also provides the use of pyrido[2,3-d]pyrimidine derivative WB3 as HIV-1 inhibitor.

The technical proposal of the invention is as follows:

1. Pyrido[2,3-d]Pyrimidine Derivative WB3

The invention provides pyrido[2,3-d]pyrimidine derivative WB3, and pharmaceutically acceptable salt, ester or prodrug thereof.

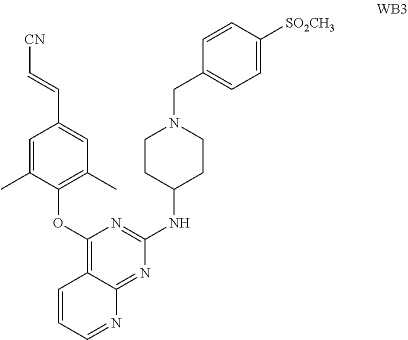 [SMILES: Cc1cc(/C=C/C#N)cc(C)c1Oc1nc(NC2CCN(Cc3ccc(S(C)(=O)=O)cc3)CC2)nc2ncccc12]

2. Preparation of Pyrido[2,3-d]Pyrimidine Derivative WB3

The preparation method of pyrido[2,3-d]pyrimidine derivative WB3 is as follows: Firstly, the starting material 2,4-dichloropyrido[2,3-d]pyrimidine (1) was reacted with (E)-3-(4-hydroxy-3,5-dimethylphenyl)acrylonitrile afforded intermediate 2. Then 2 was treated with N-(tert-butoxycarbonyl)-4-aminopiperidine yielded the intermediate 3, which was followed treated by trifluoroacetic acid afforded the key intermediate 4. Then, compound 4 was reacted with 4-(bromomethyl)benzenesulfonamide to give the target compound WB3.

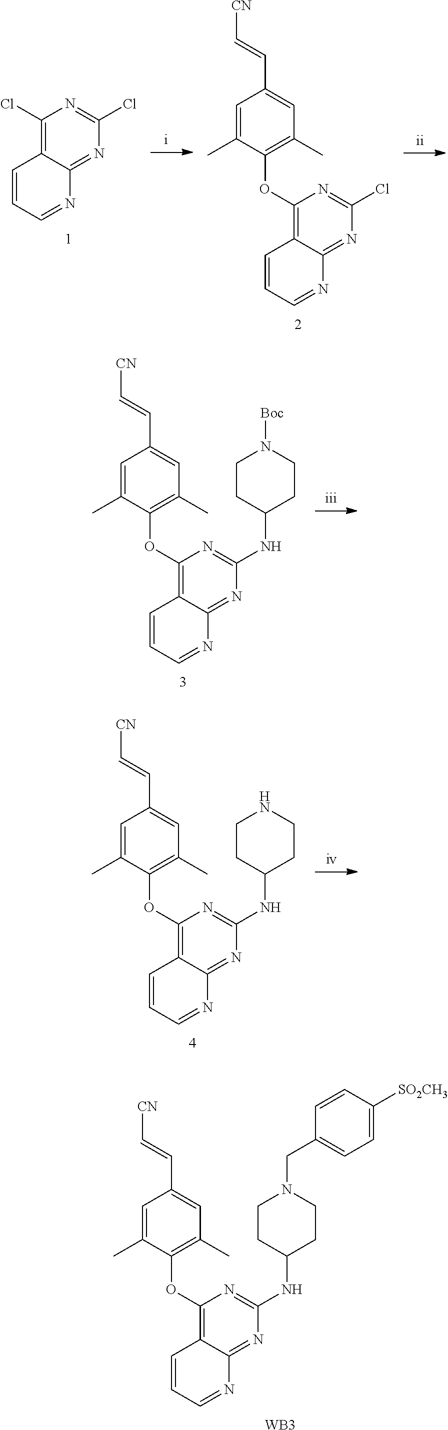

Reagents and conditions: (i) (E)-3-(4-hydroxy-3,5-dimethylphenyl)acrylonitrile, DMF, K₂CO₃, 25° C.; (ii) 4-(tert-butoxycarbonyl)aminopiperidine, DMF, K₂CO₃, 100° C.; (iii) TFA, DCM, 25° C.; (iv) 1-(bromomethyl)-4-(methylsulfonyl)benzene, DMF, K₂CO₃, 25° C.

3. Activity Evaluation and Application of Pyrido[2,3-d]Pyrimidine Derivative WB3

The target compound WB3 was evaluated its activity against HIV-1 NL4-3, HIV-1 mutant strains GH9 (K101P+K103N+V108I), RV1 (K101E+Y181V), RV2 (K101E+Y181C+G190A), RV3 (K101E+Y181C+E138K), RV4 (L100I+M230I) and RV5 (M230L). Rilpivirine (RPV) was selected as control drug.

The values of EC₅₀ (anti-HIV activity) and CC₅₀ (cytotoxicity) was depicted in Table 1. WB3 exhibited potent activity against these HIV-1 strains. For wild-type HIV-1 NL4-3 strain, WB3 was demonstrated with an EC₅₀ value of 1.16 nM. In the case of mutant strains GH9, RV1, RV2, RV3, RV4, and RV5, WB3 also displayed prominent potency. Especially for the NNRTI-resistant strain GH9, WB3 exhibited an EC₅₀ value of 1.37 nM, which was up to 200-fold potent than that of rilpivirine (EC₅₀>273 nM).

Then, WB3 was evaluated its pharmacokinetics profiles by single iv and po administration in Wistar rats. As shown in Table 2, the results showed that WB3 has moderate clearance rate (CL=20.6 mL/min/kg) and short half-life (T_{1/2}=1.59 h) when administered at 2 mg/kg intravenously, and the maximum concentration (Cₘₐₓ) was 1488 ng/mL. The assessment of WB3 at a dose of 20 mg/kg orally indicated that it was rapidly absorbed with Tₘₐₓ value of 0.417 h, Cₘₐₓ value of 2354 ng/mL and a half-life of 1.77 h. It is gratifying that the oral bioavailability (F) of WB3 reaches up to 59.9%. These favorable PK results are sufficient to support the ability of WB3 to become an orally bioavailable NNRTIs drug candidate.

Also described here is pyrido[2,3-d]pyrimidine derivative WB3 used as HIV-1 NNRTIs, furthermore, it can be used as anti-AIDS drug candidate.

Also described here are pharmaceutical composition comprising pyrido[2,3-d]pyrimidine derivative WB3, and with one or more kind of pharmaceutically acceptable carrier or excipient.

The present invention provides a novel pyrido[2,3-d]pyrimidine derivative WB3, preparation method, anti-HIV-1 activity evaluation, pharmacokinetics study and its first application in the field of anti-HIV-1 drug discovery. The pyrido[2,3-d]pyrimidine derivative WB3 have been proved to be useful as anti-HIV-1 drug candidate and have higher application value. In particular, it can be used to prepare anti-AIDS drug.

### EXAMPLES

Selected examples are listed as follows, the invention includes these compounds disclosed herein but not confined to them.

### Example 1: The Preparation of (E)-3-(4-((2-chloropyrido[2,3-d]pyrimidin-4-yl)oxy)-3,5-dimethylphenyl)acrylonitrile (2)

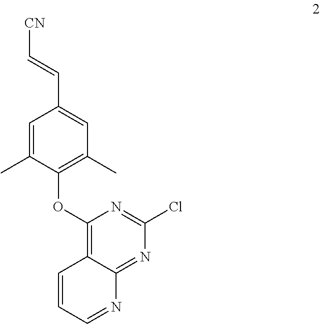 [SMILES: Cc1cc(/C=C/C#N)cc(C)c1Oc1nc(Cl)nc2ncccc12]

A reaction mixture of 2,4-dichloropyrido[2,3-d]pyrimidine (2.2 g, 10 mmol), potassium carbonate (1.7 g, 12 mmol), and (E)-3-(4-hydroxy-3,5-dimethylphenyl)acrylonitrile (1.73 g, 10 mmol) in DMF (20 mL) was stirred at 25° C. for 4 h. Then ice water (20 mL) was added to the mixture and the resulting precipitated solid was filtrated, washed with saturated brine (3×5 mL), and finally recrystallized from DMF/water to provide the intermediate 2 as a white solid with 86% yield. ¹H NMR (400 MHz, DMSO-d₆) δ 9.31 (dd, J=4.2, 2.0 Hz, 1H, C₅-pyridopyrimidine-H), 8.94 (dd, J=8.2, 1.9 Hz, 1H, C₇-pyridopyrimidine-H), 7.87 (dd, J=8.4, 4.5 Hz, 1H, C₆-pyridopyrimidine-H), 7.71-7.59 (m, 1H, ArCH═), 7.56 (s, 2H, C₃,C₅-Ph-H), 6.49 (d, J=16.7 Hz, 1H, ═CHCN), 2.12 (s, 6H, CH₃×2). ESI-MS: m/z 337.2 (M+1). C₁₈H₁₃ClN₄O (336.08).

### Example 2: The preparation of (E)-3-(3,5-dimethyl-4-((2-(piperidin ylamino)pyrido[2,3-d]pyrimidin-4-yl)oxy)phenyl)acrylonitrile (4)

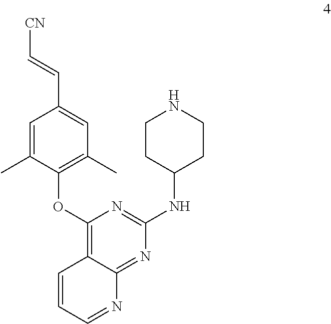 [SMILES: Cc1cc(/C=C/C#N)cc(C)c1Oc1nc(NC2CCNCC2)nc2ncccc12]

Compound 2 (1.06 g, 3.17 mmol), N-Boc-4-aminopiperidine (0.83 g, 3.80 mmol) and potassium carbonate (0.87 g, 6.33 mmol) were added in DMF (20 mL), and the mixture was stirred for 8 h at 100° C. After cooling to room temperature, the reaction mixture was slowly poured into 30 mL of ice water. The obtained precipitate was collected by filtration and dried to give crude product 3, which was used directly in the next step without further purification. Compound 3 (1.28 g, 2.53 mmol) was dissolved in a mixed solution of DCM (6 mL) and TFA (2.30 mL, 30 mmol), and the solution was stirred at room temperature for 4 h. Then the resulting liquid was alkalized to pH 9 with saturated sodium bicarbonate solution and washed with saturated brine (3×5 mL), and the aqueous phase was extracted with dichloromethane (3×10 mL). The combined organic phase was dried over anhydrous Na₂SO₄, filtered and concentrated under reduced pressure. The crude residue was further purified by flash column chromatography to afford the key intermediate 4 as a white solid with 39% yield. ¹H NMR (400 MHz, DMSO-d₆) δ 8.88 (d, J=4.3 Hz, 1H, C₅-pyridopyrimidine-H), 8.54 (d, J=8.0 Hz, 1H, C₇-pyridopyrimidine-H), 7.63 (dd, J=16.0, 7.1 Hz, 2H, C₆-pyridopyrimidine-H, ArCH═), 7.52 (s, 2H, C₃,C₅-Ph-H), 7.39-7.23 (m, 1H, NH), 6.45 (d, J=16.5 Hz, 1H, ═CHCN), 4.12 (s, 1H, piperidine-H), 3.28 (s, 1H), 3.03 (t, J=12.5 Hz, 2H, piperidine-H), 2.11 (s, 6H, CH₃×2), 2.04 (d, J=14.6 Hz, 2H, piperidine-H), 1.91-1.09 (m, 4H, piperidine-H). ESI-MS: m/z 401.4 [M+1]⁺. C₂₃H₂₄N₆O (400.20).

### Example 3: The Preparation of WB3

Compound 4 (0.20 g, 0.5 mmol), 1-(bromomethyl)-4-(methylsulfonyl)benzene (0.15 g, 0.6 mmol) and anhydrous K₂CO₃ (0.14 g, 1.0 mmol) were added to anhydrous DMF (10 mL), and the reaction mixture was stirred at room temperature for 4 h (monitored by TLC). Then water (30 mL) was added, and the mixture was extracted with ethyl acetate (3×10 mL). The organic phase was dried over anhydrous Na₂SO₄ and purified by flash column chromatography to obtain the target compound WB3. White solid in 75% yield. ¹H NMR (400 MHz, DMSO-d₆) δ 8.84 (d, J=4.3 Hz, 1H, C₅-pyridopyrimidine-H), 8.49 (d, J=7.9 Hz, 1H, C₇-pyridopyrimidine-H), 7.87 (d, J=7.9 Hz, 2H, C₃,C₅-Ph′-H), 7.63 (s, 1H, ArCH═), 7.57 (d, J=8.0 Hz, 2H, C₂,C₆-Ph′-H), 7.50 (s, 2H, C₃,C₅-Ph″-H), 7.39 (d, J=7.9 Hz, 1H, NH), 7.25 (dd, J=8.1, 4.6 Hz, 1H, C₆-pyridopyrimidine-H), 6.43 (d, J=16.7 Hz, 1H, ═CHCN), 3.85 (d, J=10.6 Hz, 1H, piperidine-H), 3.57 (s, 2H, N—CH₂), 3.20 (s, 3H, SO₂CH₃), 2.79 (d, J=11.1 Hz, 2H, piperidine-H), 2.10 (s, 6H, CH₃×2), 1.99 (s, 1H, piperidine-H), 1.82 (d, J=12.2 Hz, 2H, piperidine-H), 1.66-1.35 (m, 2H, piperidine-H), 1.18 (t, J=7.1 Hz, 1H, piperidine-H). ¹³C NMR (100 MHz, DMSO-d₆) δ 166.1, 162.4, 159.9, 157.4, 151.8, 150.3, 145.4, 139.8, 133.5, 131.9, 131.5, 129.8, 128.7, 127.4, 119.3, 118.3, 105.4, 97.0, 61.9, 52.8, 48.5, 44.0, 40.6, 40.4, 40.2, 40.0, 39.7, 39.5, 39.3, 31.5, 16.6. ESI-MS: m/z 569.5 [M+1]⁺. C₃₁H₃₂N₆O₃S (568.22).

### Example 4. In Vitro Anti-HIV Activity

Inhibition of HIV-1 infection was measured as reduction in luciferase gene expression after a single round of virus infection of TZM-bl cells. Briefly, 200 TCID₅₀ of virus (NL4-3, GH9, RV-1, RV-2, RV-3, RV-4 and RV-5) was used to infect TZM-bl cells in the presence of various concentrations of compounds. Two days after infection, the culture medium was removed from each well and 100 μL of Bright Glo reagent (Promega, Luis Obispo, Calif.) was added to the cells for measurement of luminescence with a Victor 2 luminometer. The EC₅₀ against HIV-1 strains was defined as the concentration that caused a 50% reduction of luciferase activity (relative light units) compared to virus control wells. The cytotoxicity of compounds was determined using a colorimetric 2,3-bis-(2-methoxy-4-nitro-5-sulfophenyl)-2H-tetrazolium-5-carboxanilide (XTT) assay. An amount of 100 μL of the test compounds at graded concentrations was added to equal volumes of cells (5×105/mL) in wells of a 96-well plate. Incubation at 37° C. for four days, then 50 μL of XTT solution (1 mg/mL) containing 0.02 μM phenazine methosulfate (PMS) was added. After four hours, the absorbance at 450 nm was measured with an ELISA reader. The CC₅₀ values were calculated using the CalsuSyn computer program as described above. The 50% cytotoxic concentration (CC₅₀) was defined as the concentration of the test compound that reduced the viability of the mock-infected TZM-bl cells by 50%. The 50% effective concentration (EC₅₀) was defined as the concentration of the test compound achieving 50% protection from the cytopathic effect of the virus in infected cells.

TABLE 1
The activity and cytotoxicity of WB3 and RPV

| | EC₅₀ (nM)^{a} | | | | | | | |
|---|---|---|---|---|---|---|---|---|
| Compound | NL₄₋₃ | GH9 | RV-1 | RV-2 | RV-3 | RV-4 | RV-5 | CC₅₀ (nM)^{b} |
| WB3 | 1.16 ± 0.44 | 1.37 ± 0.49 | 18.3 ± 5.80 | 29.0 ± 10.9 | >106 | 9.32 ± 2.46 | 6.15 ± 2.29 | >44.0 |
| RPV | 0.96 ± 0.30 | >273 | 45.6 ± 12.6 | 24.3 ± 5.73 | 88.1 ± 37.4 | 9.82 ± 2.35 | 10.4 ± 3.27 | >341 |

^{a}EC₅₀: concentration of compound that causes 50% inhibition of viral infection and determined in at least triplicate against HIV-1 virus in TZM-bl cell lines. NL4-3 is wild-type HIV-1 viral strain.
^{b}CC₅₀: concentration that is cytopathic to 50% of cells. The highest concentration of the tested compounds was 125 ng/mL.

### Example 5. Pharmacokinetics Assays

Ten male Wistar rats (180-200 g) were randomly divided into two groups to receive intravenous (2 mg·kg⁻¹) or oral administration (20 mg·kg⁻¹) of the test compounds. A solution of WB3 was prepared by dissolving in a mixture of polyethylene glycol (peg) 400/normal saline (70/30, V/V) before the experiment. Blood samples of the intravenous group were collected from the sinus jugular is into heparinized centrifugation tubes at 5 min, 15 min, 30 min, 1 h, 2 h, 4 h, 6 h and 10 h after dosing, and blood samples of the oral administration group were collected at 15 min, 30 min, 1 h, 2 h, 4 h, 6 h, 10 h and 12 h after dosing (200 μL of blood each times). All the samples were then centrifuged at 8000 rpm for 8 min to separate plasma, which was stored at −78° C. until analysis. LC-MS/MS analysis was used to determine the concentration of WB3 in plasma. Briefly, 50 μL of plasma was added to 50 μL of internal standard and 300 μL of methanol in a 5 mL centrifugation tube, which was centrifuged at 3000 g for 10 min. The supernatant layer was collected and a 20 μL aliquot was injected for LC-MS/MS analysis. Standard curves for WB3 in blood were generated by the addition of various concentrations of WB3 together with internal standard to blank plasma. Then, all samples were quantified with an Agilent 1200 LC/MSD (Agilent, USA). The mobile phase was methanol/1.5% glacial acetic acid (50:50, V/V) at a flow rate of 1.0 mL/min and the test wavelength was 225 nm. All blood samples were centrifuged in an Eppendorf 5415D centrifuge and quantified by Agilent 1200 LC/MSD (Agilent, USA).

The plasma pharmacokinetic data were analyzed by using the non-av model of DAS 2.0 pharmacokinetic program. The main pharmacokinetic parameters (Cₘₐₓ, AUC, Tₘₐₓ, T_{1/2}, and CL) were calculated. The results are shown in Table 2.

TABLE 2
Pharmacokinetics evaluation of WB3

| | T_{1/2} | Tₘₐₓ | Cₘₐₓ | AUC₀₋ₗₐₛₜ | AUC_{0-inf} | CL | F |
|---|---|---|---|---|---|---|---|
| subject | (h) | (h) | (ng/mL) | (h · ng/mL) | (h · ng/mL) | (mL/ | (%) |
| WB3 (iv) | 1.59 ± 0.667 | — | 1488 ± 307 | 1661 ± 368 | 1675 ± 383 | 20.6 ± 4.50 | — |
| WB3 (po) | 1.77 ± 0.822 | 0.417 ± 0.144 | 2354 ± 306 | 9934 ± 1271 | 10033 ± 1223 | — | 59.9 |

What is claimed is:

1. A pyrido[2,3-d]pyrimidine HIV-1 reverse transcriptase inhibitor WB3 is pyrido[2,3-d]pyrimidine derivative WB3, and pharmaceutically acceptable salt, ester or prodrug thereof, which has a structure as shown below:

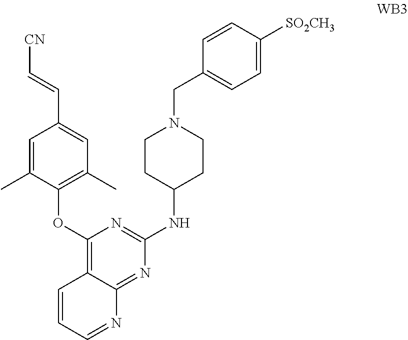 [SMILES: Cc1cc(/C=C/C#N)cc(C)c1Oc1nc(NC2CCN(Cc3ccc(S(C)(=O)=O)cc3)CC2)nc2ncccc12]

2. The pyrido[2,3-d]pyrimidine derivative WB3 of claim 1 is characterized in that the pharmaceutical acceptable salts of the compound are hydrochloride, sulfate, tartrate, citrate, and the pharmaceutical acceptable prodrugs or derivatives of WB3.

3. The pyrido[2,3-d]pyrimidine derivative WB3 of claim 1 is characterized in that the spectrum data are as follows:
¹H NMR (400 MHz, DMSO-d₆) δ 8.84 (d, J=4.3 Hz, 1H, C₅-pyridopyrimidine-H), 8.49 (d, J=7.9 Hz, 1H, C₇-pyridopyrimidine-H), 7.87 (d, J=7.9 Hz, 2H, C₃,C₅-Ph′-H), 7.63 (s, 1H, ArCH═), 7.57 (d, J=8.0 Hz, 2H, C₂,C₆-Ph′-H), 7.50 (s, 2H, C₃,C₅-Ph″-H), 7.39 (d, J=7.9 Hz, 1H, NH), 7.25 (dd, J=8.1, 4.6 Hz, 1H, C₆-pyridopyrimidine-H), 6.43 (d, J=16.7 Hz, 1H, ═CHCN), 3.85 (d, J=10.6 Hz, 1H, piperidine-H), 3.57 (s, 2H, N—CH₂), 3.20 (s, 3H, SO₂CH₃), 2.79 (d, J=11.1 Hz, 2H, piperidine-H), 2.10 (s, 6H, CH₃×2), 1.99 (s, 1H, piperidine-H), 1.82 (d, J=12.2 Hz, 2H, piperidine-H), 1.66-1.35 (m, 2H, piperidine-H), 1.18 (t, J=7.1 Hz, 1H, piperidine-H);
¹³C NMR (100 MHz, DMSO-d₆) δ 166.1, 162.4, 159.9, 157.4, 151.8, 150.3, 145.4, 139.8, 133.5, 131.9, 131.5, 129.8, 128.7, 127.4, 119.3, 118.3, 105.4, 97.0, 61.9, 52.8, 48.5, 44.0, 40.6, 40.4, 40.2, 40.0, 39.7, 39.5, 39.3, 31.5, 16.6. ESI-MS: m/z 569.5 [M+1]⁺ and C₃₁H₃₂N₆O₃S (568.22).

4. The pyrido[2,3-d]pyrimidine derivative WB3 of claim 1 is characterized by the reaction route as follows:

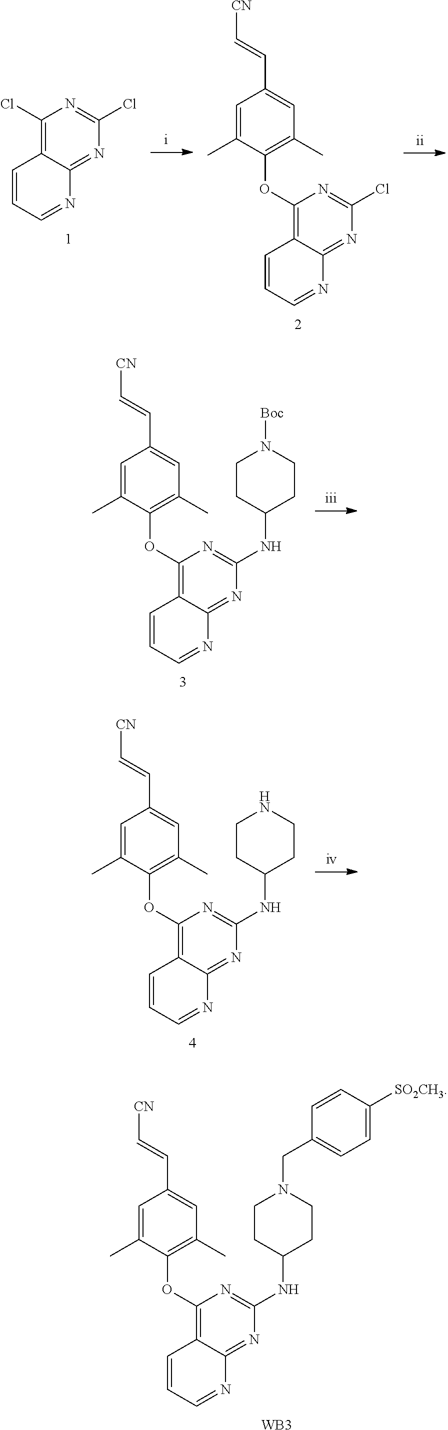

5. A preparation method of pyrido[2,3-d]pyrimidine derivative WB3 described in claim 4 is characterized by the reaction route, reagents and conditions as follows:
(i) (E)-3-(4-hydroxy-3,5-dimethylphenyl)acrylonitrile, DMF, K₂CO₃, 25° C.; (ii) 4-(tert-butoxycarbonyl)aminopiperidine, DMF, K₂CO₃, 100° C.; (iii) TFA, DCM, 25° C.; (iv) 1-(bromomethyl)-4-(methylsulfonyl)benzene, DMF, K₂CO₃, 25° C.

6. The preparation method of pyrido[2,3-d]pyrimidine derivative WB3 described in claim 4 is characterized by the following steps:
firstly, the starting material 2,4-dichloropyrido[2,3-d]pyrimidine (1) was reacted with (E) (4-hydroxy-3,5-dimethylphenyl)acrylonitrile afforded intermediate 2; then 2 was treated with N-(tert-butoxycarbonyl)-4-aminopiperidine yielded the intermediate 3, which was followed treated by trifluoroacetic acid afforded the key intermediate 4; and, compound 4 was reacted with 4-(bromomethyl)benzenesulfonamide to give the target compound WB3.

7. The pyrido[2,3-d]pyrimidine derivative WB3 of claim 1, wherein the pyrido[2,3-d]pyrimidine derivative WB3 is used for preparation of drugs for treatment and prevention of HIV.

8. The pyrido[2,3-d]pyrimidine derivative WB3 of claim 1, wherein the pyrido[2,3-d]pyrimidine derivative WB3 is used for treating HIV.

9. The pyrido[2,3-d]pyrimidine derivative WB3 of claim 1, wherein the pyrido[2,3-d]pyrimidine derivative WB3 is used for preventing HIV.

10. A pharmaceutical composition consisting of pyrido[2,3-d]pyrimidine derivative WB3 described in claim 1.

11. The pharmaceutical composition consisting of pyrido[2,3-d]pyrimidine derivative WB3 described in claim 10 and its pharmaceutically acceptable vector.

12. The pharmaceutical composition consisting of pyrido[2,3-d]pyrimidine derivative WB3 described in claim 10 and its medically acceptable excipients.

13. The pyrido[2,3-d]pyrimidine derivative WB3 of claim 1, wherein the treatment of HIV, including the pyrido[2,3-d]pyrimidine derivative WB3 is used for preparing a drug.

14. The pyrido[2,3-d]pyrimidine derivative WB3 of claim 1, wherein the pyrido[2,3-d]pyrimidine derivative WB3 is used for treating AIDS in combination with NRTIs as the main component of HAART therapy.
